(19) United States
(12) Patent Application Publication
(10) Pub. No.: US 2019/0078047 A1
(43) Pub. Date: Mar. 14, 2019

(54) CELL CULTURE APPARATUS

(71) Applicant: Panasonic Corporation, Osaka (JP)

(72) Inventors: TAKESHI ANDO, Kyoto (JP); TOSHIAKI YAMAUCHI, Kyoto (JP); NORIHIRO SHIBATA, Osaka (JP)

(21) Appl. No.: 16/189,382

(22) Filed: Nov. 13, 2018

Related U.S. Application Data
(63) Continuation-in-part of application No. 15/327,648, filed on Jan. 19, 2017.

(30) Foreign Application Priority Data
Dec. 19, 2014 (JP) 2014-257357

(51) Int. Cl. C12M 1/34; C12M 1/36; G02B 21/36; G06T 7/00

(52) U.S. Cl. CPC C12M 41/36; C12M 41/48; G06T 2207/10056; G06T 7/0012; G06T 2207/30101; G02B 21/365

(57) ABSTRACT

A cell culture apparatus includes a measurement-position designating unit, an image processing unit, and a subculture-timing determining unit. The measurement-position designating unit designates measurement positions in the culture vessel. The image processing unit calculates a confluent rate as a proportion of an area occupied by the cells in the observation image, of the positions designated by the measurement-position designating unit. The subculture-timing determining unit determines the subculture timing of the culture vessel, based on the confluent rate calculated in the image processing unit. More specifically, the subculture-timing determining unit determines the subculture timing, based on a first threshold value with respect to an average value of the entire measurement area on the observation image, and a second threshold value with respect to at least one measurement area on the observation image. The second threshold value is larger than the first threshold value.

### CROSS-REFERENCE TO RELATED APPLICATIONS

The present application is a continuation in part of U.S. patent application Ser. No. 15/327,648 filed on Jan. 19, 2017, which is a U.S. national stage application of the PCT International Application No. PCT/JP2015/004488 filed on Sep. 4, 2015, which claims the benefit of foreign priority of Japanese patent application No. 2014-257357 filed on Dec. 19, 2014, the contents all of which are incorporated herein by reference.

### TECHNICAL FIELD

The present invention relates to a cell culture apparatus that automatically determines a subculture timing.

### BACKGROUND ART

In a culture of cells, since the cells are spread over an entire culture vessel through cell proliferation, a subculture operation needs to be performed every few days. In a technology in the related art, image processing is performed on a cell observation image by a microscope or the like, and thereby a proportion of an area in the observation image, which is occupied by cells, is calculated. The subculture is performed at a timing at which the proportion exceeds a predetermined threshold value (for example, see PTL 1).

In addition, there is a cell culture apparatus in which cells at a plurality of positions in the culture vessel are observed, and which causes the culture vessel to oscillate in consideration of variations (for example, see PTL 2).

### CITATION LIST

### Patent Literature

PTL 1: Japanese Patent Unexamined Publication No. 2003-116530

PTL 2: Japanese Patent Unexamined Publication No. 2010-99011

### SUMMARY OF THE INVENTION

The present invention provides a cell culture apparatus in which it is possible to determine an appropriate subculture timing, based on image information.

A cell culture apparatus of the present disclosure including:

a measurement-position designating unit that designates a center area and a plurality of peripheral areas in a culture vessel as measurement positions;

an image processing unit that calculates a confluent rate of the center area, a confluent rate of each of the plurality of peripheral areas, and an average confluent rate which is an average of sum of the confluent rate of the center area and the confluent rates of the plurality of peripheral areas, the confluent rate being defined as a proportion of an area occupied by cells in a designated area; and

a subculture-timing determining unit that determines a timing to perform a subculture of the culture vessel, based on the average confluent rate,

wherein the subculture-timing determining unit determines the timing to perform the subculture when the average confluent rate is smaller than a first threshold value, the confluent rate of the center area is larger than a second threshold value, and the second threshold value is larger than the first threshold value.

According to the present invention, it is possible to determine an appropriate subculture timing.

### BRIEF DESCRIPTION OF DRAWINGS

FIG. 1 is a schematic diagram illustrating a cell culture apparatus according to an exemplary embodiment of the present invention.

FIG. 2 is a diagram illustrating an observation image of cells according to the exemplary embodiment of the present invention.

FIG. 3 is a graph illustrating a relationship between a confluent rate and a period of time in the exemplary embodiment of the present invention.

FIG. 4A is a view illustrating measurement positions in the exemplary embodiment of the present invention.

FIG. 4B is a view illustrating examples of measurement positions in the exemplary embodiment of the present invention.

FIG. 5A is a view illustrating a first uneven-spread state in the exemplary embodiment of the present invention.

FIG. 5B is a view illustrating a second uneven-spread state in the exemplary embodiment of the present invention.

FIG. 6 is a view illustrating a process of a subculture-timing determining unit in the exemplary embodiment of the present invention.

### DESCRIPTION OF EMBODIMENT

Before an exemplary embodiment of the present invention is described, a problem of a cell culture apparatus in the related art is briefly described.

In a case of determining a subculture timing as in PTL 1, a state of an entire culture vessel is estimated from an observation image taken at one position; however, the cells rarely grow evenly in the vessel during a cell culture, and thus there is a possibility that it is difficult to determine the subculture timing with accuracy.

In addition, in a case where an operation of the cell culture apparatus is determined, based on the measurement at a plurality of positions of the culture vessel as in PTL 2, the easiness of distribution of the cells in the culture vessel is not considered and thus there is a possibility that a difference will be found in a result of the measurement at positions through a selected method.

Hereinafter, the exemplary embodiment of the present invention will be described with reference to the accompanying figures.

Note that the same components are assigned with the same reference signs and thus description thereof is omitted in some cases.

In addition, the figures schematically illustrate the components as main bodies for easy understanding.

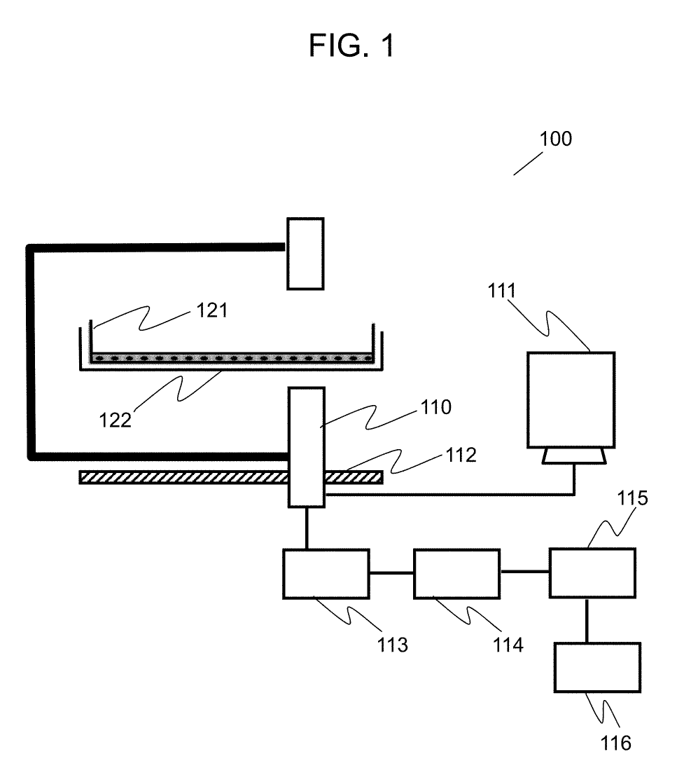

FIG. 1 is a schematic diagram illustrating cell culture apparatus 100 according to the exemplary embodiment of the present invention.

Cell culture apparatus 100 calculates a confluent rate in a plurality of measurement positions in culture vessel 121, and thereby it is possible to determine a subculture at the optimum subculture-timing in consideration of uneven spread of the cells. Here, the subculture indicates an operation of dissemination of the proliferated cells in culture vessel 121 to another culture vessel, in order to prevent the number of cells in culture vessel 121 from excessively increasing.

As illustrated in FIG. 1, cell culture apparatus 100 includes mounting unit 122, observing unit 110, measurement-position designating unit 111, drive unit 112, image measuring unit 113, image processing unit 114, time-based recording unit 115, and subculture-timing determining unit 116. Culture vessel 121 is mounted on mounting unit 122. Observing unit 110 observes cells in culture vessel 121. Measurement-position designating unit 111 designates one or more measurement positions which are observed by observing unit 110. Drive unit 112 causes observing unit 110 to move to the measurement positions designated by measurement-position designating unit 111. Image measuring unit 113 captures and records observation images at positions designated by measurement-position designating unit 111. Image processing unit 114 calculates a proportion of areas occupied by the cells in the images measured by image measuring unit 113. Time-based recording unit 115 records discrete variations with hour in the confluent rate of the cell calculated by image processing unit 114. Subculture-timing determining unit 116 determines the subculture timing, based on an image processing result at the plurality of positions designated in measurement-position designating unit 111.

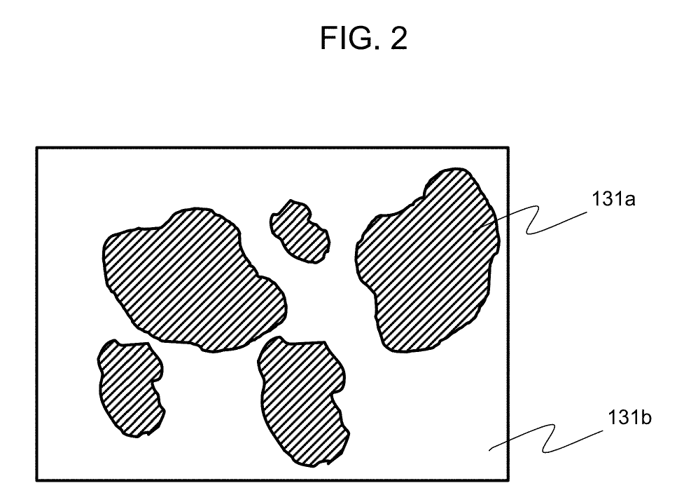

Here, the confluent rate of the cells is a proportion of specific areas of the cell portions in the observation image, to the entire area of the observation image. In the specific description with reference to FIG. 2, the entire area of the observation image is an area including hatched region 131a and unhatched region 131b, and the specific area of the cell portions is the area of hatched region 131a. In a word, in FIG. 2, hatched region 131a schematically shows the specific area of the cell portions.

A general image processing method can be used for specifying the area of the cell portions in FIG. 2. For example, template matching method is used. In this case, image processing unit 114 stores an image of cells to be specified as a template in advance, and performs matching for the template in the observation image. Then, the area of matching is defined as the specific area of the cell portions, i.e., hatched region 131a, and a proportion of areas occupied by the cells in the observation images is calculated.

Observing unit 110 is an imaging device which takes an image or a video image of cells in culture vessel 121. Observing unit 110 is, for example, a CCD (Charge Coupled Device) camera or a CMOS (Complementary Metal Oxide Semiconductor) camera or the like.

Measurement-position designating unit 111 is an interface capable of setting a measurement-position. Measurement-position designating unit 111 is, for example, a keyboard for inputting data into a computer or a touch panel or the like.

Drive unit 112 is, for example, a driving motor or the like.

Image measuring unit 113 is an imaging device including an image sensor. Image measuring unit 113 is, for example, a CCD (Charge Coupled Device) camera or a CMOS (Complementary Metal Oxide Semiconductor) camera or the like.

Image processing unit 114 is a program executing part for image processing to calculate the confluent rate. Image processing unit 114 is, for example, a CPU (Central Processing Unit) or a processor or the like. The image processing unit 114 can execute, for example, an image processing software or the like, commonly used, is used as image processing method.

Time-based recording unit 115 is a recording medium for recording the confluent rate. Time-based recording unit 115 is, for example, a memory or the like.

Subculture-timing determining unit 116 is a program executing part for determining the subculture timing. Subculture-timing determining unit 116 is, for example, a CPU (Central Processing Unit) or a processor or the like.

Subculture-timing determining unit 116 determines the subculture timing based on average value A of the confluent rate of the cells measured at the plurality of measurement positions designated by measurement-position designating unit 111. Specifically, when the confluent rate of the cells is checked by predetermined period of time and the average value A of the confluent rate exceeds predetermined first threshold value Ap of the confluent rate, subculture-timing determining unit 116 determines it is the timing to perform the subculture. Note that first threshold value Ap is set as the confluent rate at which the cells proliferates at a low speed when the confluent rate exceeds the threshold value, and is set in consideration of the efficiency of the cell culture.

Subculture-timing determining unit 116 determines the subculture timing as illustrated later, in consideration of the uneven spread of the cell proliferation in the observation image in addition to average value A of the confluent rate. In other words, even in a case where average value A of the confluent rate is smaller than first threshold value Ap, subculture-timing determining unit 116 determines it is the timing to perform the subculture in a case where the confluent rate is locally larger than preset second threshold value Ap′ due to the uneven spread of the cells. Here, second threshold value Ap′ is a value larger than first threshold value Ap. For example, in a case where human iPS cells are cultured in an undifferentiated state, that an increase in the confluent rate causes the cells to be differentiated is known. The detection of local confluent rate and a corresponding countermeasure prevent the cells from being locally differentiated and thus it is possible to prevent the cells from being degraded. Note that, when second threshold value Ap′ exceeds the threshold value, there is a possibility that differentiated cells will be found together in an undifferentiation maintaining culture of the human iPS cells, and thus second threshold value Ap′ is set.

A case of uneven spread of the main cells occurs in two types of states including a state in which the disseminated cells unevenly spread in the central portion of culture vessel 121 due to a vortex on a culture medium generated in culture vessel 121 (hereinafter, referred to as a “first uneven-spread state”) and a state in which the disseminated cells unevenly spread in a straight line shape in culture vessel 121 due to a wave of the culture medium generated in response to acceleration and deceleration during transportation of culture vessel 121 (hereinafter, referred to as a “second uneven-spread state”).

For example, since culture vessel 121 is often caused to move such that a positional relationship between culture vessel 121, a manipulator, and the like changes, after the dissemination of the cells, the first uneven-spread state or the second uneven-spread state described above is likely to occur.

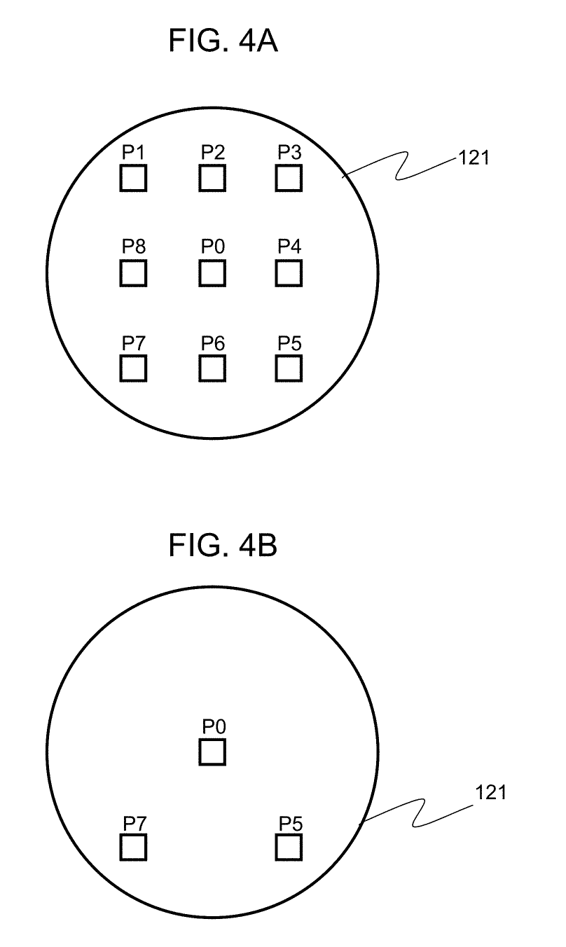

Therefore, measurement-position designating unit 111 needs to set the measurement position such that the two types of phenomena are reliably checked. Specific setting is described with reference to FIGS. 4A and 4B. In the exemplary embodiment, as illustrated in FIG. 4A, a center of culture vessel 121 is set as measurement area P0, and measurement areas P1 to P8 are set at equal intervals along a side of a square at positions separated from measurement area P0 by at least a predetermined distance (2 cm in a petri dish of 10 cm). Specific culture vessel 121 used here is a vessel with a circular bottom having a diameter of 10 cm, and the measurement area is a rectangular region (3 mm×4 mm). In the case of having such setting, in order to check the phenomenon of the first uneven-spread state, at least center P0 of culture vessel 121 is set to measure one area. In addition, in order to check the phenomenon of the second uneven-spread state, P0 in the central portion and any two areas P7 and P5 other than the central portion of culture vessel 121 need to be measured.

However, in order to check the phenomenon of the second uneven-spread state, two or more areas (for example, in FIG. 4B, measurement areas P0 and P7) existing on a straight line shape of measurement areas P0 to P8 are set to be measured. In addition, to check the phenomenon of the second uneven-spread state more certainly, at least one area (for example, in FIG. 4B, measurement area P5) out of the straight line described above and two or more areas on the straight line may be compared.

When measurement-position designating unit 111 designates, as the measurement position, at least three areas illustrated in FIG. 4B since the first uneven-spread state and the second uneven-spread state described above do not simultaneously occur, it is possible to find a phenomenon in which the confluent rate locally increases.

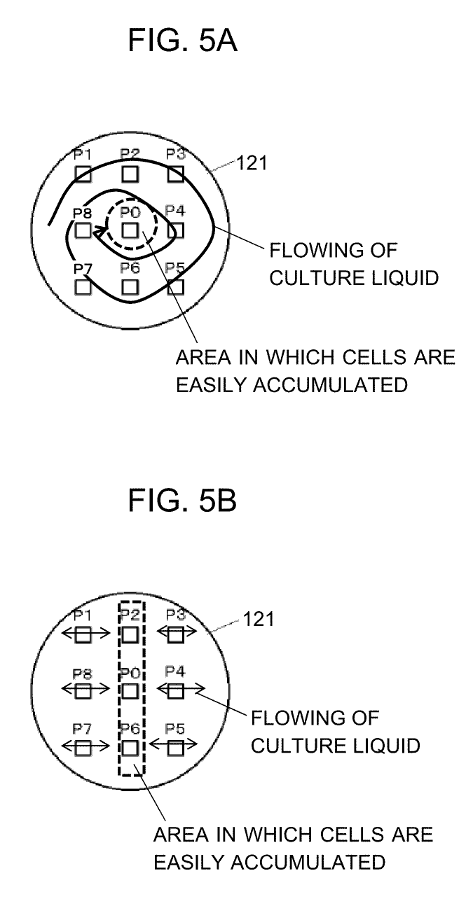

Here, a phenomenon of the first uneven-spread state and a phenomenon of the second uneven-spread state are specifically described with reference to the figures. FIG. 5A is a diagram illustrating the first uneven-spread state in the exemplary embodiment. FIG. 5B is a diagram illustrating the second uneven-spread state in the exemplary embodiment.

In the first uneven-spread state, in the process of transportation of culture vessel 121, since culture liquids flow to form a vortex shape in culture vessel 121, the cells are likely to be accumulated in measurement area P0 of the center of culture vessel 121. Meanwhile, in the second uneven-spread state, in the process of the transportation of culture vessel 121, since the culture liquids flow in a transverse wave shape in culture vessel 121, the cells are likely to be accumulated to form a straight line shape (here, measurement areas P2, P0, and P6) in culture vessel 121.

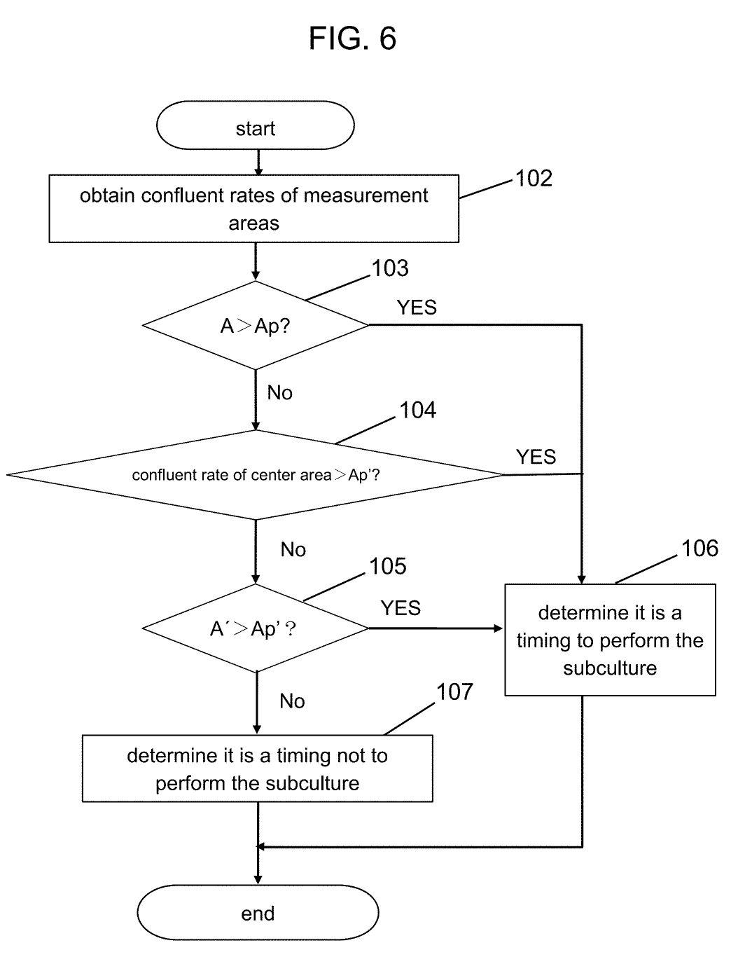

Conditions for determining the timing that the subculture is performed are more described in detail. FIG. 6 is a view illustrating a process of subculture-timing determining unit 116 in the exemplary embodiment of the present invention. Note that, “A” is an average value A of the confluent rate. Ap is a first threshold value. Ap′ is a second threshold value.

First, in a case where the nine areas (P0 to P8) described above are designated as the measurement positions, subculture-timing determining unit 116 obtains respective confluent rates of measurement areas P0-P8 obtained by image processing unit 114 (step 102). Here, subculture-timing determining unit 116 may obtain average confluent rate A and linear confluent rate A from image processing unit 114, or calculate average confluent rate A and linear confluent rate A using obtained confluent rates.

Next, subculture-timing determining unit 116 determines it is a timing to perform the subculture in a case where any one of the following three conditions is satisfied (step 106).

(1) A case where average value A of the confluent rate in all of the measurement areas of the measurement areas P0 to P8 exceeds first threshold value Ap (step 103).

(2) A case where average value A of the confluent rate in all of the measurement areas of measurement areas P0 to P8 is smaller than first threshold value Ap; however, the confluent rate of center P0 exceeds second threshold value Ap′ (step 104).

(3) A case where average value A of the confluent rate in all of the measurement areas of measurement areas P0 to P8 is smaller than first threshold value Ap; however, linear confluent rate A, which is average value of the confluent rates of two or more areas (for example, measurement areas P1, P0, and P5) existing on a straight line shape passing measurement area P0 among measurement area P0 and measurement area P1 to P8, exceeds second threshold value Ap′ (step 105).

Here, (2) above is a condition in which the first uneven-spread state described above is determined, and (3) above is a condition in which the second uneven-spread state described above is determined.

Determination of whether or not each condition is satisfied makes it possible to determine the first uneven-spread state or the second uneven-spread state. Specifically, it is also possible that a case where (1) above is not satisfied, but (2) above is satisfied, the first uneven-spread state is determined, and a case where (1) above is not satisfied, but (3) above is satisfied, the second uneven-spread state is determined. Note that, in a case where the conditions of (1) to (3) above are not satisfied, for example, in a case where only two areas (for example, measurement areas P5 and P7) which do not exist on the straight line of measurement areas P1 to P8 exceed the value of the second threshold value Ap′, or the like, the variations in a selection method of the measurement area without the uneven spread in the cell proliferation is determined, and thus subculture-timing determining unit 116 determines it is a timing not to perform the subculture (step 107). In a case where human iPS cells are cultured in colonies, and first threshold value Ap is from 45% to 70%, and second threshold value Ap′ is from 75% to 90%, it is possible to culture the cells while the cells are maintained in a stable manner without degradation. More preferably, it is possible to culture the cells while the cells are maintained in a more stable manner without degradation, in a case where first threshold value Ap is 60% and second threshold value Ap′ is 80%.

Note that, when the values of threshold values Ap and Ap′ are set to be low, the cells are maintained to have high qualities; however, the proliferation of the cells decreases and the number of divisions decreases.

Meanwhile, when the values of threshold values Ap and Ap′ are set to be high, the qualities of the cells are likely to be degraded; however, it is possible to increase the number of cells so as to increase the number of divisions. In other words, a mode of having an emphasis on quality of the cells in the culture and a mode of having an emphasis on the number of the cells are set, and values of threshold values Ap and Ap′ are smaller than normal in a case where the mode of having an emphasis on quality is selected. The values of threshold values Ap and Ap′ are larger than normal in a case where the mode of having an emphasis on the number is selected, and thereby it is possible to perform the subculture in which the quality or the number of cells is emphasized.

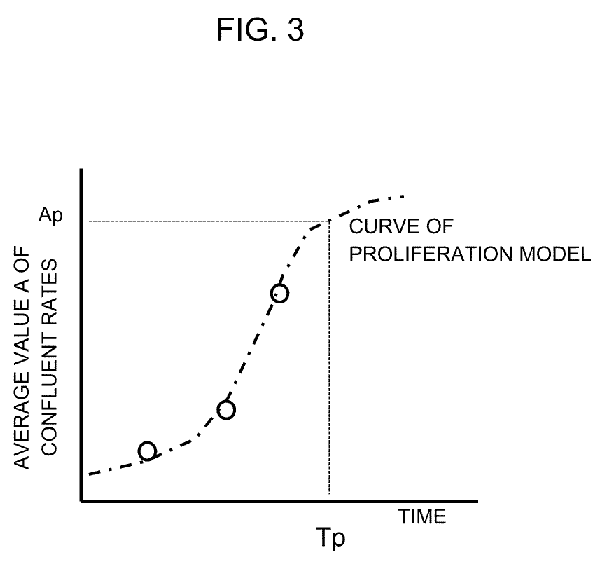

Note that it is possible to estimate a period of subculture time by calculating an approximate cell growth curve illustrated in FIG. 3 on the basis of a cell proliferation model set depending on types of cells designated in advance and a change in the confluent rate recorded in time-based recording unit 115 and then estimating a time point Tp at which the confluent rate reaches predetermined first threshold value Ap. In this manner, it is possible to perform the subculture at a more appropriate timing, and thus it is possible to realize stability in the quality of the cells.

Note that, in the culture vessel having the confluent rate that exceeds the second threshold value, there is a possibility that the confluent rate will locally increase and degraded cells will be mixed therein. Therefore, the cell culture apparatus is further provided with a culture vessel information control unit that controls information of captured images, date and time of capturing, captured measurement area, and the like of the culture vessel, and that adds a graph to information as data associated with a culture vessel to a culture vessel having the confluent rate exceeding the second threshold value. The graph shows that the confluent rate exceeds the second threshold value. In a case where a user operates the culture vessel, a message indicating that the confluent rate exceeds the second threshold value is displayed on a display unit disposed in the cell culture apparatus, and it is possible to notify the user of the message.

Note that, in a case where the cells proliferate not in a single cell state, but in the colony as the human iPS cells, image processing unit 114 can not only calculate the confluent rate of the cells, but also calculate the diameter of the colony. In the case of the human iPS cells, the cells are known to be differentiated in a case where the colony has a very large diameter. It is preferable that subculture-timing determining unit 116 determines the subculture timing on the basis of the confluent rate and distribution the colonies having diameters in the plurality of measurement positions. In this manner, it is possible to perform culture with high accuracy with quality maintained.

A part of components or all components included in cell culture apparatus 100 in the embodiment described above may be controlled by one or a plurality of control units. Here, the control unit is structured by one system LSI (Large Scale Integration) or a plurality of dedicated electronic circuits or the like. For example, the control unit accepts an input from measurement-position designating unit 111, and executes processing operated by image processing unit 114 and subculture-timing determining unit 116, then determines subculture timing. The system LSI is a multi-function LSI manufactured by integrating a plurality of components on a chip. Specifically, the system LSI is a computer system structured by a microprocessor, ROM (Read Only Memory), RAM (Random Access Memory) or the like. The ROM stores a computer program, and the microprocessor is operated according to the computer program, thus, the system LSI performs its function.

### INDUSTRIAL APPLICABILITY

The cell culture apparatus of the present invention is applicable to a regenerative medicine and a drug discovery field.

1. A cell culture apparatus comprising:
a measurement-position designating unit that designates a center area and a plurality of peripheral areas in a culture vessel as measurement positions;
an image processing unit that calculates a confluent rate of the center area, a confluent rate of each of the plurality of peripheral areas, and an average confluent rate which is an average of sum of the confluent rate of the center area and the confluent rates of the plurality of peripheral areas, the confluent rate being defined as a proportion of an area occupied by cells in a designated area; and
a subculture-timing determining unit that determines a timing to perform a subculture of the culture vessel, based on the average confluent rate,
wherein the subculture-timing determining unit determines the timing to perform the subculture when the average confluent rate is smaller than a first threshold value, the confluent rate of the center area is larger than a second threshold value, and the second threshold value is larger than the first threshold value.

2. The cell culture apparatus according to claim 1,
wherein, in a case where a square whose center is a point included in the center area is defined, the plurality of peripheral areas are eight areas including four vertex points at the square and four middle points of four sides at the square.

3. The cell culture apparatus according to claim 2,
wherein, the center area and the plurality of peripheral areas are separated from each other.

4. The cell culture apparatus according to claim 3,
wherein the plurality of peripheral areas are the eight areas whose centers are the four vertex points at the square and the four middle points of the four sides at the square.

5. The cell culture apparatus according to claim 1,
wherein the first threshold value is from 45% confluent rate to 70% confluent rate, both inclusive, and the second threshold value is from 75% confluent rate to 90% confluent rate, both inclusive.

6. The cell culture apparatus according to claim 5,
wherein the first threshold value is 60% confluent rate, and the second threshold value is 80% confluent rate.

7. A cell culture apparatus comprising:
a measurement-position designating unit that designates a center area and a plurality of peripheral areas in a culture vessel as measurement positions;
an image processing unit that calculates a confluent rate of the center area, a confluent rate of each of the plurality of peripheral areas, and an average confluent rate which is an average of sum of the confluent rate of the center area and the confluent rates of the plurality of peripheral areas, the confluent rate being defined as a proportion of an area occupied by cells in a designated area; and
a subculture-timing determining unit that determines a timing to perform a subculture of the culture vessel, based on the average confluent rate,
wherein in a case where a line which connects two points included in two areas among the plurality of peripheral areas and a point included in the center area shows a straight line, the image processing unit that calculates a linear confluent rate which is an average of sum of the confluent rate of the center area and the confluent rates of the two points, and
the subculture-timing determining unit determines the timing to perform the subculture when the average confluent rate is smaller than a first threshold value, the linear confluent rate is larger than a second threshold value, and the second threshold value is larger than the first threshold value.

8. The cell culture apparatus according to claim 7,
wherein, in a case where a square whose center is a point included in the center area is defined, the plurality of peripheral areas are eight areas including four vertex points at the square and four middle points of four sides at the square.

9. The cell culture apparatus according to claim 8,
wherein the center area and the plurality of peripheral areas are separated from each other.

10. The cell culture apparatus according to claim 9,
wherein the plurality of peripheral areas are the eight areas whose centers are the four vertex points at the square and the four middle points of the four sides at the square.

11. The cell culture apparatus according to claim 7,
wherein the first threshold value is from 45% confluent rate to 70% confluent rate, both inclusive, and the second threshold value is from 75% confluent rate to 90% confluent rate, both inclusive.

12. The cell culture apparatus according to claim 11,
wherein the first threshold value is 60% confluent rate, and the second threshold value is 80% confluent rate.
